(19) United States
(12) Patent Application Publication
(10) Pub. No.: US 2024/0240228 A1
(43) Pub. Date: Jul. 18, 2024

(54) BRET-BASED CORONAVIRUS MPRO PROTEASE SENSOR AND USES THEREOF

(71) Applicant: Qatar Foundation for Education, Science and Community Development, Doha (QA)

(72) Inventors: Anupriya M. Geethakumari, Doha (QA); Kabir Hassan Biswas, Doha (QA)

(21) Appl. No.: 18/562,772

(22) PCT Filed: May 20, 2022
(86) PCT No.: PCT/QA2022/050009

§ 371 Date: Nov. 20, 2023

Related U.S. Application Data
(60) Provisional application No. 63/275,217, filed on Nov. 3, 2021.
(60) Provisional application No. 63/191,788, filed on May 21, 2021.

(51) Int. Cl. C12Q 1/37; G01N 21/64

(52) U.S. Cl. CPC C12Q 1/37; G01N 21/6428; G01N 21/6458

(57) ABSTRACT

The SARS-CoV-2 main protease, M^{PRO}, is critical for its replication and is an appealing target for designing anti-SARS-CoV-2 agents. In this regard, a number of assays have been developed based on its cleavage sequence preferences to monitor its activity. These include the usage of Fluorescence Resonance Energy Transfer (FRET)-based substrates in vitro and a FlipGFP reporter, one which fluoresces after M^{PRO}-mediated cleavage, in live cells. Here, a pair of genetically encoded, Bioluminescence Resonance Energy Transfer (BRET)-based sensors have been engineered for detecting SARS-CoV-2 M^{PRO} proteolytic activity in living host cells. The sensors were generated by sandwiching M^{PRO} N-terminal autocleavage sites, either AVLQSGFR (short) or KTSAVLQSGFRKME (long), in between the mNeonGreen and nanoLuc proteins. Co-expression of the sensor with the M^{PRO} in live cells resulted in its cleavage in a dose-dependent manner while mutation of the critical C145 residue (C145A) in M^{PRO} completely abrogated the sensor cleavage. A temporal activity of M^{PRO} in live cells and its inhibition was shown using the well-characterized pharmacological agent GC376. The sensor developed here finds direct utility in studies related to drug discovery targeting the SARS-CoV-2 M^{PRO} and functional genomics application to determine the effect of sequence variation in M^{PRO} Importantly, the BRET-based sensors displayed increased sensitivities and specificities as compared to the recently developed FlipGFP-based M^{PRO} sensor. Additionally, the sensors recapitulated the inhibition of M^{PRO} by the well-characterized pharmacological agent GC376. Further, in vitro assays with the BRET-based M^{PRO} sensors revealed a molecular crowding-mediated increase in the rate of M^{PRO} activity and a decrease in the inhibitory potential of GC376. The sensor developed here finds direct utility in studies related to drug discovery targeting the SARS-CoV-2 M^{PRO} and functional genomics application to determine the effect of sequence variation in M^{PRO}.

### CROSS REFERENCE TO RELATED APPLICATIONS

This application is a national phase application under section 371 of International Patent Application PCT/QA2022/050009, filed May 20, 2022, which claims the benefit of U.S. provisional patent application No. 63/275,217, filed Nov. 3, 2021, and U.S. provisional patent application 63/191,788, filed May 21, 2021, the entire disclosures, each of which are incorporated herein by reference.

### FIELD

Described herein are Bioluminescence Resonance Energy Transfer (BRET)-based coronavirus protease activity sensors, and methods of using the same to detect the activity of coronavirus proteases or coronavirus inhibitors.

### SEQUENCE LISTING

A Sequence Listing is submitted herewith and incorporated by reference herein as an XML file created on Oct. 23, 2023, entitled “5600234-01086_Sequence_Listing.xml” and having a size of 9 KB.

### BACKGROUND

Coronaviruses are a large family of viruses that usually cause mild to moderate upper-respiratory tract illnesses, like the common cold. However, three new coronaviruses have emerged from animal reservoirs over the past two decades that cause serious and widespread illness and death: severe acute respiratory syndrome coronavirus (SARS-CoV); Middle East respiratory syndrome coronavirus (MERS-CoV); and severe acute respiratory syndrome coronavirus 2 (SARS-CoV-2). SARS-CoV-2 causes the disease, coronavirus disease 2019 (COVID-19). COVID-19 has become a global health threat with more than 50 million infections and 1 million deaths. Coronaviruses envelop positive-stranded ribonucleic acid (RNA) that, when released into a cell, is translated into two overlapping polyproteins, pp1a and pp1ab. Main chymotrypsin-like protease (known as M^{pro}, 3CL^{pro}, or nsp5) auto-cleaves itself from within these polyproteins- and cleaves the remaining polyproteins into the viral machinery required to control viral replication in the infected cell. Therefore, M^{pro} is recognized as critical for viral replication and a target for designing anti-SARS-n-2 agents.

### SUMMARY

Disclosed herein are sensors comprising J¹ and J². In some embodiments, the sensors comprise J¹ and J² which are connected by a linker. In some embodiments, the linker can be an M^{pro} peptide sequence. In some embodiments, J¹ comprises a NanoLuc peptide sequence. In some embodiments, J² comprises an mNeonGreen peptide sequence. In some embodiments, J¹ comprises a NanoLuc peptide sequence, and J² comprises a mNeonGreen peptide sequence. The M^{pro} peptide sequence can comprise an M^{pro} cleavage peptide sequence.

In some embodiments, disclosed herein are methods of determining M^{pro} proteolytic inhibition of a compound. In some embodiments, a method of determining M^{pro} proteolytic inhibition of a compound comprises contacting a compound with an M^{pro} peptide sequence having protease activity in the presence of a sensor.

In other embodiments, disclosed herein are methods of determining protease activity of an M^{pro} peptide sequence. In some embodiments, a method of determining protease activity of an M^{pro} peptide sequence comprises contacting a sensor with the M^{pro} peptide sequence.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows a schematic representation of the genetically encoded, BRET-based SARS-CoV-2 M^{pro} protease activity sensor expressed in live cells. The M^{pro} cleavage site (AVLQSGFR) is SEQ ID NO:1. Close positioning of the NLuc (BRET donor) and mNG (BRET acceptor) proteins result in a significant resonance energy transfer in the absence of the SARS-CoV-2 M^{pro} protease activity. Activity of the SARS-CoV-2 M^{pro} protease results in the cleavage of the sensor resulting in a decrease in the resonance energy transfer between NLuc and mNG resulting in a decrease in the green fluorescence of the sensor.

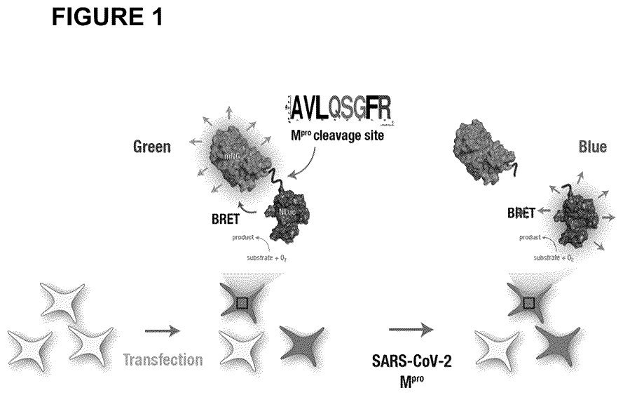

FIGS. 2A-B show cleavage of the M^{pro} sensor constructs in live cells. The schematics show the M^{pro} sensor constructs-short (FIG. 2A; SEQ ID NO:1) and long (FIG. 2B; SEQ ID NO:2)—with SARS418 CoV-2 M^{pro} N-terminal autocleavage sequence. FIGS. 2C-D illustrate graphs showing bioluminescence spectra of the short (FIG. 2C) and long (FIG. 2D) M^{pro} sensor constructs either in control cells or in cells expressing the WT or C145A mutant M^{pro} protease. Data were fit to a two Gaussian model reflecting mNG fluorescence and NLuc bioluminescence peaks. A reduction was observed in the mNG peak (533 nm) of both the short and the long sensors when co-expressed with the wild type 423 M^{pro}, while no reduction was observed when co-expressed with the C145A mutant M^{pro}. FIGS. 2E-F illustrate graphs showing total mNG fluorescence (measured prior to substrate addition) in cells expressing the short (FIG. 2E) and the long (FIG. 2F) sensors. FIG. 2G illustrates a graph showing BRET ratio (ratio emission at 533 nm and 467 nm) of the short (left side) and the long (right side) M^{pro} protease activity sensors in either control cells or when co-expressed with the wild type or the C145A mutant M^{pro} protease. The inset graph of FIG. 2G shows the percentage change in BRET of the short (left side) and the long (right side) when co-expressed with the wild type or the C145A mutant M^{pro} protease. The top panel of FIG. 2H illustrates an anti-His tag blot showing cleavage of the short (left side) and the long (right side) M^{pro} sensor constructs in either control cells or in cells co-expressing the wild type or the C145A mutant M^{pro} protease. There was a release of an approximately 30 kDa, His₆-tagged-mNG fragment in cells expressing the wild type, but not in the C145A mutant M^{pro} protease. The bottom panel of FIG. 2H illustrates an anti-Strep-tag blot showing expression of the M^{pro} protease in the respectively transfected cells.

FIGS. 3A-D show M^{pro} protease DNA dose-dependent cleavage of the M^{pro} sensors in live cells. FIGS. 3A-B illustrate graphs showing bioluminescence spectra of the short (FIG. 3A) and long (FIG. 3B) M^{pro} sensor constructs in cells transfected with the indicated amounts of either the WT or the C145A mutant M^{pro} protease plasmid DNA. Data were fit to a two Gaussian model reflecting mNG fluorescence and NLuc bioluminescence peaks. FIGS. 3C-D illustrate graphs showing BRET ratio of the short (FIG. 3C) and the long (FIG. 3D) M^{pro} sensors in cells transfected with the indicated amounts of either the wild type or the C145A mutant M^{pro} protease plasmid DNA. The inset graphs of FIGS. 3C-D show the percentage decrease in BRET ratio compared to the control cells when transfected with the indicated amounts of the wild type M^{pro} protease plasmid DNA.

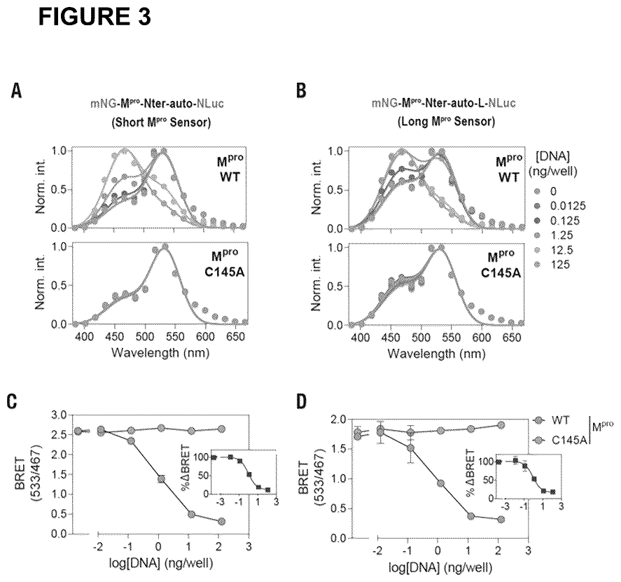

FIGS. 4A-D show temporal dynamics of M^{pro} protease activity in live cells. FIGS. 4A-B illustrate graphs showing bioluminescence spectra of the short (FIG. 3A) and long (FIG. 3B) M^{pro} sensor constructs at the indicated times post transfection in either control cells or cells transfected with the WT or the C145A mutant M^{pro} protease. Data were fit to a two Gaussian model reflecting mNG fluorescence and NLuc bioluminescence peaks. A time-dependent decrease in the mNG fluorescence (533 nm peak) in cells transfected with wild type was noted, but not in the C145A mutant M^{pro} protease. FIGS. 2C-D illustrate graphs showing the BRET ratio of the short (FIG. 4C) and the long (FIG. 4D) M^{pro} sensors at the indicated time post transfection in either control cells or cells transfected with the wild type or the C145A mutant M^{pro} protease. The insets graphs of FIGS. 4C-D show the percentage change in BRET ratio compared to the control cells with time when transfected with the wild type or mutant M^{pro}.

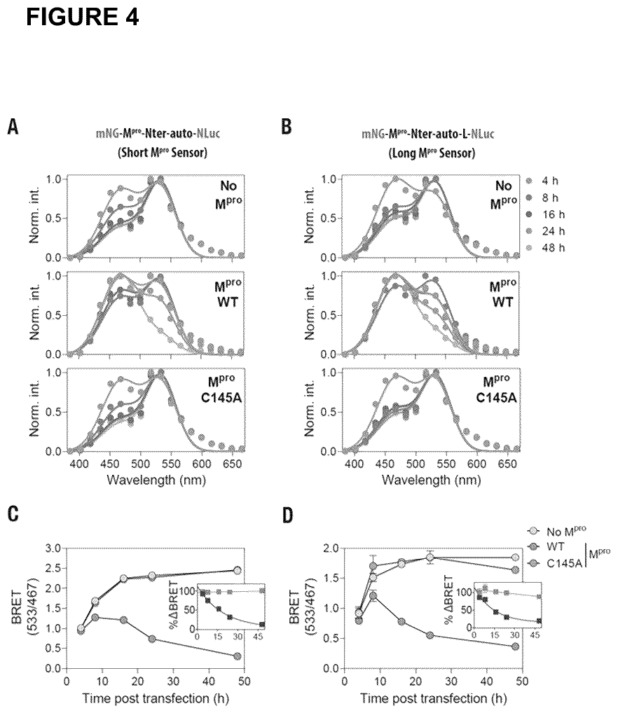

FIGS. 5A-D show M^{pro} inhibition monitored in live cells. FIGS. 5A-B illustrate graphs showing bioluminescence spectra of the short (FIG. 5A) and long (FIG. 5B) M^{pro} sensor constructs in cells treated with the indicated concentrations of GC376 inhibitor in cells co-expressing either the WT or the C145A mutant M^{pro} protease. Data were fit to a two Gaussian model reflecting mNG fluorescence and NLuc bioluminescence peaks. Gaussian plot has shown that the M^{pro} inhibitor, GC376, inhibits the protease activity at concentrations above 10 μM which is evident from the increased intensity of green fluorescence. FIGS. 5C-D represent the BRET graphs which demonstrate that the BRET ratio increases with increase in inhibitor concentration. In addition, the graphs also represent the decrease in percentage of protease activity with increase in inhibitor concentration. The BRET ratio of the mutant is not affected by GC376.

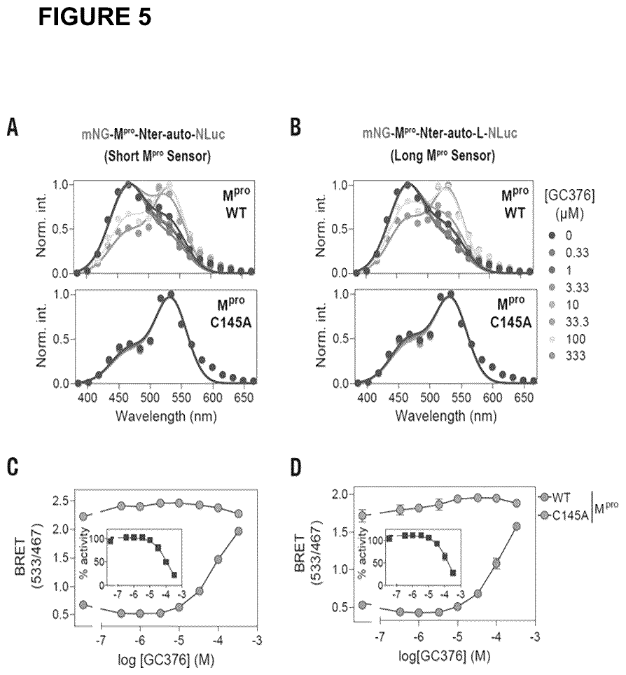

FIG. 6 shows a schematic representation of the genetically encoded, BRET-based SARS-CoV-2 M^{pro} protease activity sensor expressed in live cells. The M^{pro} cleavage site (AVLQSGFR) is SEQ ID NO:1. Close positioning of the NLuc and mNG proteins result in a significant resonance energy transfer in the absence of the SARS-CoV-2 M^{pro} protease activity. Activity of the SARS-CoV-2 M^{pro} protease results in the cleavage of the sensor resulting in a decrease in the resonance energy transfer between NLuc and mNG resulting in a decrease in the green fluorescence of the sensor.

FIGS. 7A-J show the M^{pro} N-terminal autocleavage peptide. The schematics show the M^{pro}-Nter-auto (short; represented by FIG. 7A) and M^{pro}-Nter-auto-L (long; represented by FIG. 7B) peptide structures modeled using the peptide substrate crystallized with H41A mutant SARS-CoV M^{pro} (PDB: 2Q6G). FIGS. 7C-D illustrate graphs showing backbone (Cα) root-mean-square deviation (RMSD) values of M^{pro}-Nter-auto (short; represented by FIG. 7C) and M^{pro}-Nter-auto-L (long; represented by FIG. 7D) peptide obtained from 1 μs of Gaussian MD simulations. FIGS. 7E-F illustrate graphs showing backbone (Cα) root-mean-square fluctuation (RMSF) values of M^{pro}-Nter-auto (short; represented by FIG. 7E) and M^{pro}-Nter-auto-L (long; represented by FIG. 7F) peptides. FIGS. 7G-H illustrate graphs showing radius of gyration (Rg) of the M^{pro}-Nter-auto (short; represented by FIG. 7G) and M^{pro}-Nter-auto-L (long; represented by FIG. 7H) peptides monitored over 1 μs of Gaussian MD simulations. FIGS. 7I-J illustrate graphs showing frequency of indicated secondary structures formed by the M^{pro}-Nter-auto (short [SEQ ID NO:1]; represented by FIG. 7I) and M^{pro}-Nter-auto-L (long [SEQ ID NO:2]; represented by FIG. 7J) peptides over 1 μs of Gaussian MD simulation.

FIGS. 8A-B show a secondary structure prediction of the M^{pro} BRET sensor linkers containing M^{pro} cleavage sites. FIG. 8A shows a secondary structure prediction of the short M^{pro} BRET sensor linker containing M^{pro} cleavage sites (SEQ ID NO:3). FIG. 8B shows a secondary structure prediction of the long M^{pro} BRET sensor linkers containing M^{pro} cleavage sites (SEQ ID NO:4).

FIGS. 9A-B illustrate a fluorescence image of live cells showing expression of the M^{pro} sensor. Epifluorescence images acquired using a 4× objective of HEK 293T cells transfected with either pmNG-M^{pro}-Nter-auto-NLuc (short [SEQ ID NO:3] represented by FIG. 8A) or pmNG-M^{pro}-Nter-auto-L-NLuc (long [SEQ ID NO:4] represented by FIG. 8B) plasmids showing robust expression of the sensor constructs in these cells.

FIG. 10 shows M^{pro} plasmid DNA dose-dependent cleavage of the M^{pro} sensors in live cells. FIG. 10A illustrates graphs showing bioluminescence spectra of the short M^{pro} sensor constructs in cells expressing either the WT or C145A mutant M^{pro} protease. FIG. 10B illustrates graphs showing bioluminescence spectra of the long M^{pro} sensor constructs in cells expressing either the WT or C145A mutant M^{pro} protease.

FIGS. 11A-D show M^{pro} protease DNA dose-dependent cleavage of the M^{pro} sensors in live cells. FIGS. 11A-B illustrate graphs showing bioluminescence spectra of the short (FIG. 11A) and long (FIG. 11B) M^{pro} sensor constructs at the indicated times post transfection in either control cells or cells transfected with the WT or the C145A mutant M^{pro} protease. Data were fit to a two Gaussian model reflecting mNG fluorescence and NLuc bioluminescence peaks. A time-dependent decrease in the mNG fluorescence (533 nm peak) in cells transfected with wild type was seen, but not with the C145A mutant M^{pro} protease. FIGS. 11C-D illustrate graphs showing BRET ratio of the short (FIG. 11C) and the long (FIG. 11D) M^{pro} sensors at the indicated time post transfection in either control cells or cells transfected with the wild type or the C145A mutant M^{pro} protease. The inset graphs of FIGS. 11C-D illustrate graphs showing a percentage decrease in BRET ratio compared to the control cells when transfected with the wild type or mutant M^{pro}.

FIGS. 12A-B show temporal dynamics of M^{pro} protease activity in live cells. FIGS. 12A-B represent graphs showing bioluminescence spectra of the short (FIG. 12A) and long (FIG. 12B) M^{pro} sensor constructs either in control cells or in cells expressing the WT or C145A mutant M^{pro} protease.

FIGS. 13A-D show a time-dependent expression of the BRET-based M^{pro} sensors. FIG. 13A and FIG. 13C represent epifluorescence images acquired using a 4× objective of HEK 293T cells transfected with either pmNG-M^{pro}-Nter-auto-NLuc (short; represented by FIG. 13A) or pmNG-M^{pro}-Nter-auto-L-NLuc (long; represented by FIG. 13C) plasmids showing a time-dependent increase in the number of cells expressing the sensors. FIG. 13B and FIG. 13D represent graphs showing time-dependent increase in GFP⁺ cells after transfection with either pmNG-M^{pro}-Nter-auto-NLuc (short; represented by FIG. 13B) or pmNG-M^{pro}-Nter-auto-L-NLuc (long; represented by FIG. 13D) plasmids.

FIGS. 14A-D illustrate the M^{pro} proteolytic activity using the FlipGFP-based M^{pro} sensor in live cells. FIG. 14A represents epifluorescence images of cells showing time-dependent expression of GFP, which is converted from the non-fluorescent FlipGFP upon proteolytic cleavage by M^{pro} (top panel), mCherry (middle panel) and merge (bottom panel) in cells transfected with the M^{pro} WT. FIG. 15B depicts graphs showing GFP and mCherry fluorescence in individual cells transfected with the M^{pro} WT at the indicated time points. FIG. 14C represents epifluorescence images of cells showing time-dependent expression of GFP (top panel), mCherry (middle panel) and merge (bottom panel) in cells transfected with the C145A mutant M^{pro}. FIG. 14D represents graphs showing GFP and mCherry fluorescence in individual cells transfected with the C145A mutant M^{pro} at the indicated time points.

FIGS. 15A-B show the GC376-mediated M^{pro} inhibition monitored in live cells and the bioluminescence spectra of the no M^{pro} control. FIGS. 15A-B depict graphs showing bioluminescence spectra of the short (FIG. 15A) and long (FIG. 15B) M^{pro} sensor constructs in cells treated with the indicated concentrations of GC376 inhibitor in cells co-expressing either the WT or the C145A mutant M^{pro} protease.

FIGS. 16A-B show M^{pro} inhibition monitored in live cells. FIGS. 16A-B represent graphs showing bioluminescence spectra of the short (FIG. 16A) and long (FIG. 16B) M^{pro} sensor constructs in cells treated with the indicated concentrations of GC376 inhibitor in cells co-expressing either the WT or the C145A mutant M^{pro} protease. Data were fit to a two Gaussian model reflecting mNG fluorescence and NLuc bioluminescence peaks. Gaussian plot has shown that the M^{pro} inhibitor, GC376, inhibits the protease activity at concentrations above 10 μM, which is evident from the increased intensity of green fluorescence. The BRET graph has shown that the BRET ratio increases with increase in inhibitor concentration. In addition, the graph also represents the decrease in percentage of protease activity with increase in inhibitor concentration. The BRET ratio of the mutant is not affected by GC376.

FIGS. 17A-H show the molecular crowding-mediated increase in M^{pro} proteolytic activity and decrease in GC376 potency. FIG. 17A represents a graph showing in vitro proteolytic cleavage kinetics of the short M^{pro} biosensor under the indicated concentrations of recombinantly purified SARS-CoV M^{pro} protein. FIG. 17A represents a graph showing in vitro proteolytic cleavage kinetics of the short M^{pro} biosensor in the absence and presence of 25% (w/v) of PEG 20000 (20K). FIGS. 17C-D represent graphs showing GC376-mediated inhibition of SARS-CoV M^{pro} proteolytic cleavage of the short M^{pro} sensor in the absence (FIG. 17C) and presence of 25% (w/v) of PEG 20000 (FIG. 17D). FIGS. 17E-F represent graphs showing concentration-dependent inhibition of SARS-CoV M^{pro} (FIG. 17E) and logIC₅₀ values (FIG. 17F) in the absence and presence of 25% (w/v) of PEG 20000. FIGS. 19G-H represent IC50 values of 73.1±7.4 and 86.9±11.0 nM for the short and the long sensor, respectively.

FIG. 18 depicts graphs showing frequency distribution of size (diameter, nm) of bovine serum albumin BSA (left panel) and the short M^{pro} biosensor (right panel) determined from multiple (N=40 for BSA and 61 from the short M^{pro} biosensor) dynamic light scattering (DLS) measurements. Insets in the respective graphs show a representative measurement.

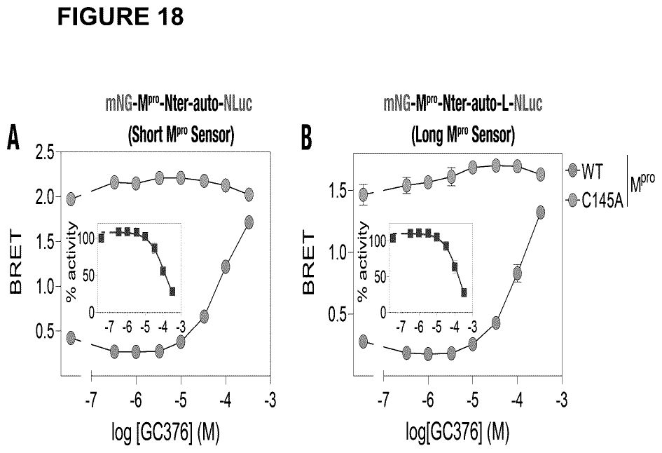

FIG. 19. In vitro enzyme kinetics assay using the short M^{pro} sensor. FIG. 19 represents a graph showing kinetic measurements of the short M^{pro} sensor cleavage in reactions containing the indicated concentrations of M^{pro}. Data plotted are average of four measurements±SD and fit to the allosteric sigmoidal equation in GraphPad Prism. A decrease in the Hill coefficient (h) at 500 nM of M^{pro} was observed.

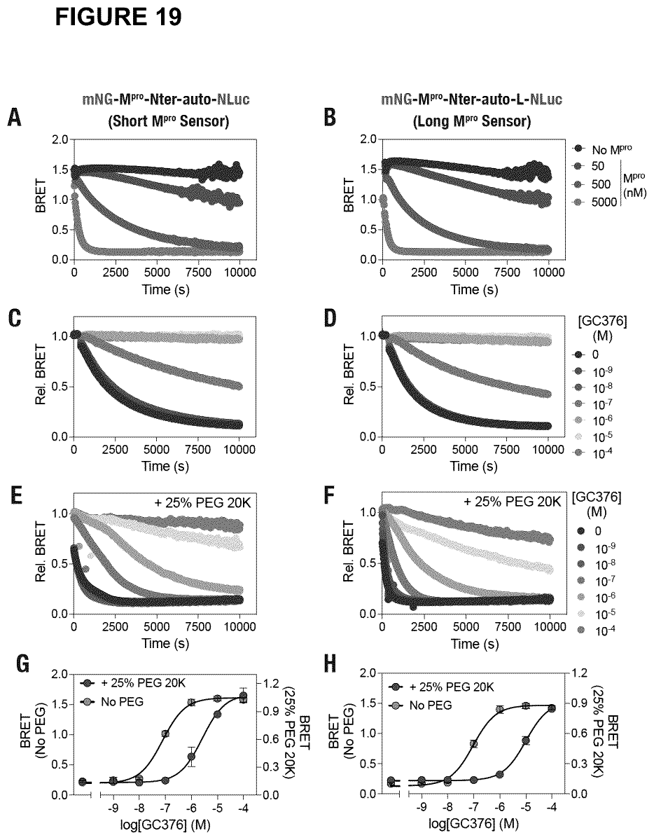

### DETAILED DESCRIPTION

The present disclosure describes a live cell-based assay to detect M^{pro} activity in a cell, and thereby a method of screening for SARS-CoV-2 therapeutics. The two reporters exemplified herein are BRET donor/acceptor pairs linked by an autocatalytic target of M^{pro} (mNeonGreen-AVLQSGFR-NanoLuc, and mNeonGreen-KTSAVLQSGFRKME-NanoLuc).

COVID-19 has become a global health threat with more than 50 million infections and 1 million deaths. The causative agent, severe acute respiratory syndrome coronavirus 2 (SARS-CoV-2) of the beta-coronavirus family shares 79% similarity with SARS-CoV and 50% similarity with MERS-CoV (Middle East respiratory syndrome coronavirus). The SARS-CoV-2 infection cycle is initiated by the processing of two polypeptides, pp1a and pp1ab, bearing the non-structural proteins by the auto-catalytically released viral proteases, 3-chymotrypsin-like cysteine protease (3CL^{pro}) or main protease (M^{pro}), and papain-like protease (PL^{pro}). M^{pro} functions as a homodimer with each monomer containing an active site formed by a conserved catalytic dyad of Cys-His, and cleaves the large polyprotein pp1ab at 11 sites. Specifically, M^{pro} recognizes a highly conserved core sequence with a critical Gln residue for cleavage. Importantly, M^{pro} cleavage sequences are not known to be recognized by human proteases, thus making M^{pro} an attractive target for anti-SARS-CoV-2 therapy.

Given the critical role played by M^{pro} in SARS-CoV-2 infection and the cleavage specificity, a number of assays have been developed to monitor the proteolytic activity of M^{pro}. Genetic reporter assays based on fluorescence and bioluminescence provide sensitive and effective systems to assess the cellular functions including cell signaling, protein dimerization, conformational changes of proteins and protein-protein interactions in live cells. Researchers have developed fluorescence and bioluminescence-based reporter assays for screening antiviral molecules against various coronaviruses (e.g. fluorescence resonance energy transfer (FRET), split-luciferase). Specifically, a number of studies have utilized FRET-based in vitro assays, wherein peptide substrates containing the M^{pro} cleavage sequences are used as reporter, for the identification of antivirals against SARS-CoV-2 M^{pro}. Additionally, a FRET-based assay was utilized for the identification of Boceprevir, GC376, and calpain inhibitors II, XII as potent inhibitors of SARS-CoV-2 M^{pro}. On the other hand, a FlipGFP-based construct containing the M^{pro} N-terminal autocleavage site has been developed to screen the antivirals against SARS-CoV-2. In such a construct, M^{pro}-mediated cleavage of FlipGFP in live cells results in the generation of the fluorescent form of GFP from the non-fluorescent form.

In addition to the above, Bioluminescence Resonance Energy Transfer (BRET) has been used in developing a range of genetically encoded, live cell sensors. BRET relies on the non-radiative resonance energy transfer from a light emitting luciferase protein (donor) upon oxidation of its substrate to a fluorescent protein (acceptor) with an excitation spectrum overlapping with the luciferase emission spectra. In addition to the spectral overlap, BRET also depends on the physical distance and relative orientation of the donor and the acceptor proteins. The latter has been successfully utilized in generating a variety of molecular sensors including detecting small molecules, structural changes in proteins. While a number of donor-acceptor pairs with distinct spectral and energy transfer efficiencies have been utilized for BRET-based sensor development, the combination of mNeonGreen (mNG), a bright green fluorescent protein, and NanoLuc (NLuc), a small bright and stable luciferase have gained significant usage in the recent times including proteolytic cleavage sensors due to excellent spectral overlap and light emission characteristics. In the present disclosure, a BRET-based M^{pro} proteolytic activity sensor was developed by inserting the M^{pro} N-terminal autocleavage sequences (either the short AVLQSGFR [SEQ ID NO:1] or the long KTSAVLQSGFRKME [SEQ ID NO:2] in between the mNG (acceptor) and the NLuc (donor) in a single fusion construct. The sensor constructs showed robust cleavage activity in live cells when co-expressed with the wild type M^{pro}, both in a dose-dependent and time-dependent manner, but not in the presence of the catalytically dead C145A mutant M^{pro}. The utility of the sensors in pharmacological inhibition of the M^{pro} was determined using the well-established M^{pro} inhibitor, GC376.

Disclosed herein are sensors comprising J¹ and J². In some embodiments, the sensors comprise J¹ and J², wherein J¹ and J² are connected by a linker. In some embodiments, the linker can be a M^{pro} peptide sequence. In some embodiments, J¹ comprises a NanoLuc peptide sequence. In some embodiments, J² comprises a mNeonGreen peptide sequence. In some embodiments, J¹ comprises a NanoLuc peptide sequence, and J² comprises a mNeonGreen peptide sequence. The M^{pro} peptide sequence can comprise an M^{pro} cleavage peptide sequence. In some embodiments, the linker can further comprise a M^{pro} protease peptide sequence.

In some embodiments, a sensor comprises J¹ connected by a linker to J², wherein the linker comprises an M^{pro} peptide sequence; J¹ comprises a NanoLuc peptide sequence; and J² comprises an mNeonGreen peptide sequence.

In some embodiments, the M^{pro} peptide sequence comprises an M^{pro} cleavage peptide sequence. The M^{pro} cleavage peptide sequence can comprise AVLQSGFR (SEQ ID NO:1). In other embodiments, the M^{pro} cleavage peptide sequence can comprise KTSAVLQSGFRKME (SEQ ID NO:2).

In some embodiments, the sensor can comprise the peptide sequence EFGTENLYAVLQSGFRGSGGS (SEQ ID NO:3). In other embodiments, the sensor can comprise the peptide sequence EFGTENLYKTSAVLQSGFRKMEGSGGS (SEQ ID NO:4).

In some embodiments, a method of forming a BRET-based M^{pro} proteolytic activity sensor comprises inserting a M^{pro} N-terminal autocleavage sequence in between an acceptor protein and a donor protein in a single fusion construct. The M^{pro} N-terminal autocleavage sequence can be a short sequence AVLQSGFR (SEQ ID NO:1). In other embodiments, the M^{pro} N-terminal autocleavage sequence can be a long sequence KTSAVLQSGFRKME (SEQ ID NO:2). The acceptor protein can be mNeonGreen (mNG) or any other suitable protein. The donor protein can be NanoLuc luciferase (NLuc) or an other suitable protein.

In some embodiments, the acceptor protein is a resonance energy acceptor protein. In other embodiments, the donor protein is a bioluminescence donor protein. In some embodiments, the acceptor protein is mNG which is a resonance energy acceptor protein, and the donor protein is NLuc, which is a bioluminescence donor protein.

In some embodiments, a method of forming a BRET-based M^{pro} proteolytic activity sensor comprises inserting a M^{pro} N-terminal autocleavage sequence in between an acceptor protein and a donor protein in a single fusion construct; wherein the M^{pro} N-terminal autocleavage sequence is AVLQSGFR (SEQ ID NO:1), the acceptor protein is mNeonGreen (mNG), and donor protein is NanoLuc luciferase (NLuc).

In other embodiments, a method of forming a BRET-based M^{pro} proteolytic activity sensor comprises inserting a M^{pro} N-terminal autocleavage sequence in between an acceptor protein and a donor protein in a single fusion construct; wherein the M^{pro} N-terminal autocleavage sequence the long sequence KTSAVLQSGFRKME (SEQ ID NO:2), the acceptor protein is mNeonGreen (mNG), and donor protein is NanoLuc luciferase (NLuc).

In some embodiments, disclosed herein are methods of determining M^{pro} proteolytic inhibition of a compound. In some embodiments, a method of determining M^{pro} proteolytic inhibition of a compound comprises contacting a compound with an M^{pro} peptide sequence having protease activity in the presence of a sensor described herein.

In some embodiments, the methods of determining M^{pro} proteolytic inhibition of a compound further comprise measuring a fluorescence emission of the sensor. The fluorescence emission of the sensor can be measured prior to contact with a compound, during contact with a compound, and/or after contact with a compound. In some embodiments, the fluorescence emission measured can be compared with an initial fluorescence emission of the sensor prior to contact with the compound.

In other embodiments, disclosed herein are methods of determining protease activity of an M^{pro} peptide sequence. In some embodiments, a method of determining protease activity of an M^{pro} peptide sequence comprises contacting a sensor described herein with the M^{pro} peptide sequence. The methods of determining protease activity of a M^{pro} peptide sequence can further comprise measuring a fluorescence emission of the sensor and comparing the fluorescence emission with an initial fluorescence emission of the sensor prior to contact with the M^{pro} peptide sequence.

In some embodiments, the BRET-based M^{pro} sensors described herein can report M^{pro} proteolytic activity at about 2 hours (h) of infection, about 3 h of infection, about 4 h of infection, about 5 h of infection, about 6 h of infection, about 7 h of infection, about 8 h of infection, about 9 h of infection, about 10 h of infection, about 11 h of infection, about 12 h of infection, 2 h of infection, 3 h of infection, 4 h of infection, 5 h of infection, 6 h of infection, 7 h of infection, 8 h of infection, 9 h of infection, 10 h of infection, 11 h of infection, 12 h of infection, between about 0.5 h of infection and about 2 h of infection, between about 2 h of infection and about 4 h of infection, between about 4 h of infection and about 6 h of infection, between about 6 h of infection and about 8 h of infection, between about 8 h of infection and about 10 h of infection, or between about 8 h of infection and 12 h of infection. Hours of infection can vary depending on the actual expression of the protease in host cells.

In some embodiments, the BRET-based M^{pro} proteolytic activity sensors described herein can be utilized for screening antivirals targeted against M^{pro}. In some embodiments, the BRET-based M^{pro} proteolytic activity sensors described herein can be utilized in detecting active SARS-CoV-2 infection. Additionally, in some embodiments, the BRET-based M^{pro} proteolytic activity sensors can be utilized for determining effects of genetic variation in the M^{pro} amino acid sequence that can arise during the evolution of the virus.

Also described herein are oligonucleotide sequences, coding for any of the sensors described herein. Provided herein, are vectors comprising the DNA sequence, coding for any of the sensors described herein.

Also provided herein are compositions comprising at least one of the sensors described herein. In some embodiments, the compositions can comprise one or more excipients. As used herein, the term “excipient” refers to physiologically compatible additives useful in preparation of a pharmaceutical composition. Examples of pharmaceutically acceptable carriers and excipients can, for example, be found in Remington's Pharmaceutical Sciences, 17^{th} Ed.

In some embodiments, the sensors provided herein are in the form of a pharmaceutically acceptable salt. As used herein, the term “pharmaceutically acceptable salt” refers to derivatives of the sensors provided herein wherein the parent sensor is modified by converting one or more of an existing acid or base moiety to its salt form. Examples of pharmaceutically acceptable salts include, but are not limited to, mineral or organic acid salts of basic residues such as amines, alkali or organic salts of acidic residues such as carboxylic acids, and the like. The pharmaceutically acceptable salts of the sensors provided herein include the conventional non-toxic salts of the parent compound formed, for example, from non-toxic inorganic or organic acids. The pharmaceutically acceptable salts of the sensors provided herein can be synthesized from the parent sensor which contains one or more basic or acidic moieties by conventional chemical methods. Generally, such salts can be prepared by combining the free acid or base forms of these sensors with a stoichiometric amount (relative to the number of moieties to be converted to a corresponding salt) of the appropriate base or acid in water or in an organic solvent, or in a mixture of the two; generally, nonaqueous media such as ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

In some embodiments, the sensors or compositions provided herein are housed within a container, optionally wherein the container reduces or blocks transmission of visible or ultraviolet light through the container. In some embodiments, the sensors, housed within the container, undergo photolysis at a slower rate as compared to a container that does not reduce or block transmission of visible or ultraviolet light. In some embodiments, the sensors, when housed within the container, have a rate of photolysis that is about zero.

Described herein, are kits comprising any one of the sensors described herein and instructions for use. The kits can be used to detect active SARS-CoV-2 infection.

### EXAMPLE

### Materials & Methods

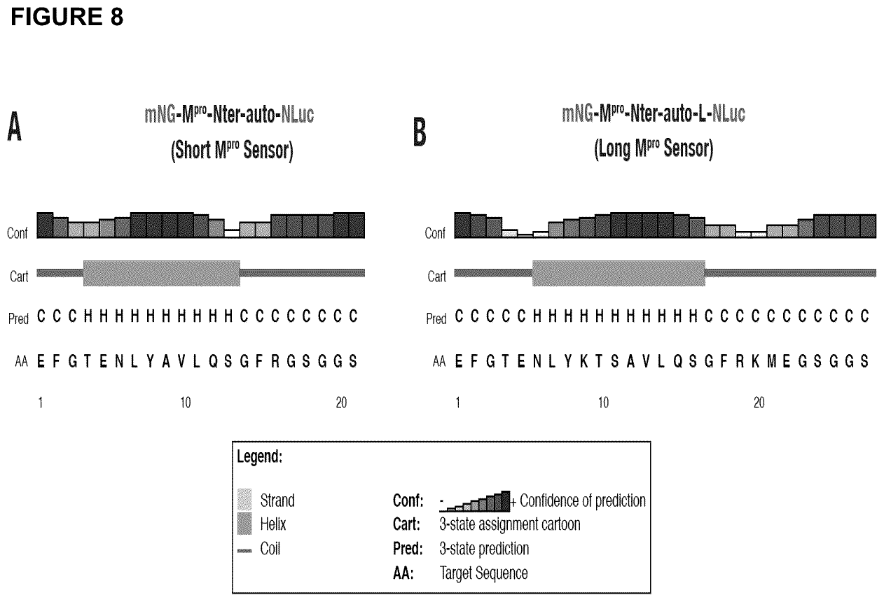

Structural modeling of M^{pro} N-terminal autocleavage peptide sequences. The crystal structure of the N-terminal peptide substrate complexed with SARS-CoV main protease H41A mutant (PDB: 2Q6G, Chain D, aa seq: TSAVLQSGFRK [SEQ ID NO:5]) was used as a template for generating the 3D models for the short and long M^{pro} cleavage peptides, including the linker region, of the MPRO sensor (short cleavage peptide aa seq: EFGTENLYAVLQSGFRGSGGS (SEQ ID NO:3) represented by FIG. 8A, long cleavage peptide aa seq: EFGTENLYKTSAVLQSGFRKMEGSGGS) (SEQ ID NO:4) represented by FIG. 8A. Models were generated using MODELLER (10.1 release, Mar. 18, 2021). Briefly, the short and long sequences were aligned with the template in PIR format. For each peptide, 100 models were initially generated using “Automodel” function and “very-slow” MD refining mode. Scoring functions such as modpdf, DOPE, and GA34, were used to assess the generated models. The model with the lowest DOPE score was further refined by loop modelling using very-slow loop MD refining mode to generate 100 refined models. The same scoring functions were used to assess the refined models. The stereochemical quality of the final model was assessed with PROCHECK.

Molecular dynamics simulation. To neutralize the positive and negative charges on the peptide's termini, the N- and C-termini were capped with N-acetyl and N-methyl amide capping groups, respectively. Topology and parameter files were generated using CHARMM-GUI webserver. The biomolecular simulation systems included the peptide model, with all hydrogens added, solvated in TIP3P (transferable interntial with 3 poimolecular potents) cubic water box with 10 Å minimum distance between edge of box and any of the peptide atoms. Charges were neutralized by adding 0.15 M NaCl to the solvated system. The total number of atoms was 15480 and 18233 for the short and long peptide simulation systems, respectively. In silico molecular dynamics simulations were performed using Nanoscale Molecular Dynamics (NAMD) software version 2.13 with the CHARMM36(m) force field. A 2 fs time-step of integration was set for all simulations performed. First, energy minimization was performed on each system for 1000 steps (2 ps). Following energy minimization, the system was slowly heated from 60 K to 310 K at 1 K interval to reach the 310 K equilibrium temperature using a temper ramp that runs 500 steps after each temperature increment. Following thermalization, temperature was maintained at 310 K using Langevin temperature control and at 1.0 atm using Nose-Hoover Langevin piston pressure control. The system was then equilibrated with 500000 steps (1 ns) using Periodic Boundary Conditions. During thermal equilibration, the peptide backbone atoms (C-CA-N) were restrained using harmonic potential to preserve the tertiary structure of the peptides. The NAMD output structure was then used as an input for GaMD simulation utilizing the integrated GaMD module in NAMD and its default parameters, which included 2 ns cMD equilibration run in GaMD, to collect potential statistics required for calculating the GaMD acceleration parameters, and another 50 ns equilibration run in GaMD after adding the boost potential, and finally GaMD production runs for 1000 ns. Both equilibration steps in GaMD were preceded by 0.4 ns preparatory runs. All GaMD simulations were run at the “dual-boost” level by setting the reference energy to the lower bound, i.e., E=Vmax. One boost potential is applied to the dihedral energetic term and the other to the total potential energetic term. The details for calculating the boost potentials including the equations used have been described previously. The upper limits of standard deviation (SD) of the dihedral and total potential boosts in GaMD were set to 6.0 kcal/mol. All GaMD simulations were performed using similar and constant temperature and pressure parameters. For all simulations, short-range non-bonded interactions were defined at 12 Å cut-off with 10 Å switching distance, while Particle-mesh Ewald (PME) scheme was used to handle long-range electrostatic interactions at 1 Å PME grid spacing. Trajectory frames were saved every 10,000 steps (20 ps) and trajectory analysis was performed using the available tools in VMD. Trajectory movies were compiled based on 1000 frames using Videomach (http://gromada.com/videomach/) to generate 41 s movies in AVI format. 2D-RMSD heatmaps were generated using MDAnalysis python toolkit.

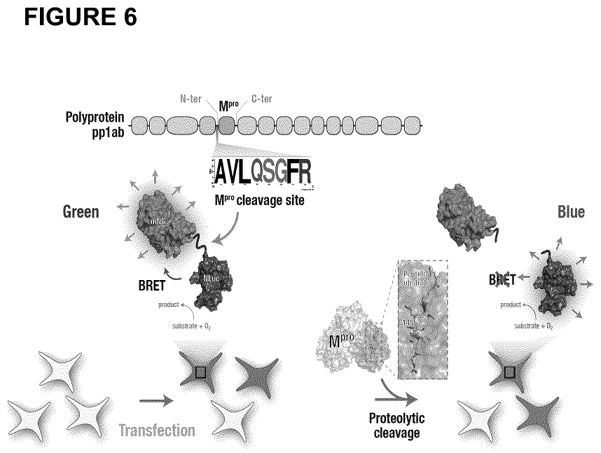

M^{pro}-terminal autocleavage sequence analysis. A total of 1984 sequences for the SARS-CoV-2 pp1a polyprotein available at the NCBI Virus database (https://www.ncbi.nlm.nih.gov/genome/viruses/) were downloaded and aligned using MAFFT server (https://mafft.cbrc.jp/alignment/server/). The aligned sequences of the pp1a polyprotein were analyzed for the conservation of the M^{pro} N-terminal autocleavage positions (AVLQSGFR) (FIG. 6).

M^{pro} BRET sensor plasmid construct generation. The BRET-based M^{pro} activity sensors were developed based on M^{pro} N-terminal autocleavage peptides, namely AVLQSGFR (SEQ ID NO:1) (nucleotide sequence 5′ GCA GTG CTC CAA AGC GGA TTT CGC 3′ [SEQ ID NO:5]) and KTSAVLQSGFRKME (SEQ ID NO:2) (nucleotide sequence 5′ AAA ACG AGT GCC GTA TTG CAG AGT GGG TTT CGG AAA ATG GAA 3′ [SEQ ID NO:7]), referred to as mNG-M^{pro}-Nter-auto-NLuc and mNG-M^{pro}-Nter-auto-L-NLuc, respectively. For these, fragments BstXI-mNG-M^{pro}-Nter-auto-NLuc-XhoI and BstXI-mNG-M^{pro}-Nter-auto-L-NLuc-XhoI were synthesized (Integrated DNA Technologies, IDT; Iowa, USA) and inserted into pIDTSmart (Kan) vectors to generate the plasmid constructs pIDT-mNG-M^{pro}-Nter-auto-NLuc and pIDT-mNG-M^{pro}-Nter-auto-L-NLuc, respectively. Both vectors were transformed into E. coli for amplification and purified using Qiagen mini-prep kit. Restriction enzymes BstX-I and XhoI were used to excise the two DNA fragments of interests from entry clones pIDT-mNG-M^{pro}-Nter-auto-NLuc and pIDT-mNG-M^{pro}-Nter-auto-L-NLuc and ligated into similarly digested destination plasmid pmNeonGreen-DEVD-NLuc [Addgene: 98287] and further confirmed by Sanger sequencing. One Shot TOP10 Competent E. coli cells were transformed with 2 μL of the ligation reaction and plated in LB agar plates with 100 μg/mL Ampicillin. Positive clones were isolated, amplified, and confirmed by the presence of inserts by Sanger sequencing using a pair of forward and reverse primers, 5′ GCACAGCCAGAACCACATATACCTT 3′ (SEQ ID NO:8) and 5′ CACCACCTTGAAGATCTTCTCGATCT 3′ (SEQ ID NO:9), respectively.

Cell culture and transfection. All experiments were performed with HEK 293T cells, which were grown in Dulbecco's Modified Eagle Medium (DMEM) supplemented with 10% fetal bovine serum, and 1% penicillin-streptomycin and grown at 37° C. in 5% CO₂. Transfections were performed with polyethyleneimine (PEI) lipid according to manufacturers' protocol. Briefly, HEK 293T cells were seeded onto 96-well white plates before 24 h of transfection. The plasmid DNA (sensor and M^{pro}), Opti-MEM (Invitrogen; 31985088) and 1.25 μg/well of PEI lipid (Sigma-Aldrich; 408727-100 mL) were combined using pipetting and incubated at room temperature for 30 minutes before being added to cells by droplet. The PEI stock solution of 2 mg/mL was prepared by diluting in sterile Milli-Q water and stored at −80° C.

Live cell, BRET-based M^{pro} proteolytic cleavage activity assays. Live cell M^{pro} proteolytic cleavage activity assays were performed by co-transfecting HEK 293T cells with either the pmNG-M^{pro}-Nter-auto-NLuc or the pmNG-M^{pro}-Nter-auto-L-NLuc M^{pro} sensor plasmid constructs along with either pLVX-EF1alpha-SARS-CoV-2-nsp5-2×Strep-IRES-Puro (M^{pro} WT) (Addgene plasmid #141370; http://n2t.net/addgene:141370; RRID:Addgene_141370) or pLVX-EF1alpha-SARS-CoV-2-nsp5-C145A-2×Strep-IRES-Puro (C145A mutant M^{pro}) plasmid (Addgene plasmid #141371; http://n2t.net/addgene:141371; RRID:Addgene_141371) in 96-well white flat bottom plates (Nunc; 136101). For dose-response experiments, the filler plasmid (a pcDNA3.1-based plasmid) is also co-transfected. In case of time-course experiment, a pcDNA3.1-based plasmid was used as a control (no M^{pro}). The time-course experiments were carried out at 1:5 reporter-to-protease ratio. Post 48 h (or otherwise indicated) of transfection, BRET measurements were performed by the addition of furimazine (Promega, Wisconsin, USA) at a dilution of 1:200. In time-course experiments, BRET was measured at the indicated time points. Experiments were performed in triplicates and repeated a minimum of two times.

Live cell, FlipGFP-based M^{pro} proteolytic assay. For live cell FlipGFP-based M^{pro} proteolytic activity assays, HEK 293T cells were seeded onto 24-well plates and co-transfected with the FlipGFP sensor plasmid (pcDNA3 FlipGFP(Mpro) T2A mCherry; provided by Xiaokun Shu; Addgene plasmid #163078) and either the WT or the C145A mutant M^{pro} expressing plasmid DNA (1.25 μg/well) using PEI lipid after 24 h of cell seeding. For transfection, cells were imaged using a EVOS FL microscope (Life Technologies; 4′ objective) at the indicated time in the red (to monitor mCherry expression to determine transfected cells) and the green (to monitor conversion of non-fluorescent FlipGFP into the fluorescent GFP form after M^{pro}-mediated cleavage) channels. Using an ImageJ macro script, images were analyzed for percentage GFP positive (GFP⁺) cells, number of transfected cells and total number of analyzed cells for each time point using Fiji. For determining number of GFP⁺ cells, GFP intensities obtained for each cell was background corrected and threshold was applied.

Cell lysate preparation for in vitro BRET assays. To prepare cell lysates containing the M^{pro} sensors, HEK 293T cells were transfected with either the mNG-M^{pro}-Nter-auto-NLuc or the mNG-M^{pro}-Nter-auto-L-NLuc M^{pro} BRET sensor and washed with chilled Dulbecco's Phosphate-Buffered Saline (DPBS) 48 h post transfection. Cells were lysed in a buffer containing 50 mM HEPES (pH 7.5), 50 mM NaCl, 0.1% Triton-X 100, 1 mM Dithiothreitol (DTT) & 1 mM ethylenediamine tetraacetic acid (EDTA) on ice. Cell lysates were collected in a 1.5 mL Eppendorf tube and centrifuged at 4° C. for 1 h at 14,000 rotations per min (RPM) following which supernatant were collected and stored at −80° C. until further usage.

In vitro, BRET-based M^{pro} proteolytic cleavage activity assays. In vitro BRET-based M^{pro} proteolytic cleavage activity assays were performed by incubating cell lysates containing the short, BRET-based M^{pro} sensor with different concentrations (0.5, 5, 50 and 500 nM) of recombinantly purified SARS-CoV M^{pro} (SARS coronavirus, 3CL Protease, Recombinant from E. coli; NR-700; BEI Resources, NIAID, NIH; stock solution of the protein was prepared by dissolving the lyophilized protein in 50 μM in Tris-buffered saline (TBS) containing 10% glycerol) and BRET monitored through luminescence scans. The effect of molecular crowding was monitored by incubating the sensor and the protease in the absence or presence of 25% (w/v) of polyethylene glycol (PEG) of a range (0.4, 2, 4, 8, 20 or 35 kDa) of molecular weights (Sigma-Aldrich). GC376 (GC376 Sodium; AOBIOUS-A0B36447; stock solution prepared in 50% DMSO at a concentration of 10 mM) inhibition of M^{PRO} protease (50 nM) was monitored under a range of the inhibitor concentrations in the absence or presence of 25% (w/v) PEG 8K. BRET measurements were performed at 37° C. by the addition of furimazine (Promega, Wisconsin, USA) at a dilution of 1:200. The bioluminescence (467 nm) and fluorescence (533 nm) readings were recorded using Tecan SPARK multimode microplate reader and used to calculate the BRET ratios (533 nm/467 nm). Total mNG fluorescence in cell lysates containing the short, BRET-based M^{pro} sensor was measured by exciting the samples at 480 nm and emission acquired at a wavelength of 530 nm.

BRET and fluorescence measurements. BRET measurements were performed using a Tecan SPARK® multimode microplate reader. Bioluminescence spectral scan was performed from 380 nm to 664 nm wavelengths with an acquisition time of 400 ms for each wavelength to determine relative emissions from NLuc (donor) and mNG (acceptor) and quantify BRET, which is expressed as a ratio of emissions at 533 nm and 467 nm. In some experiments, BRET measurements were performed by measuring emission only at 533 and 467 nm. Total mNG fluorescence in the sensor expressing cells was measured by exciting the samples at 480 nm and emission acquired at a wavelength of 530 nm.

Live cell M^{pro} proteolytic cleavage inhibitor assay. HEK 293T cells were co-transfected with either pmNG-M^{pro}-Nter-auto-NLuc or pmNG-M^{pro}-Nter-auto-L-NLuc plasmid along with either pLVX-EF1alpha-SARS-CoV-2-nsp5-2×Strep-IRES-Puro (M^{pro} WT) (Addgene plasmid #141370) or pLVX-EF1alpha-SARS-CoV-2-nsp5-C145A-2×Strep-IRES-Puro (M^{pro} C145A) (Addgene plasmid #141371) plasmid in 96-well white flat bottom plates. A 1:5 reporter-to-protease ratio was used for the assay. Eight hours post-transfection, GC376 (GC376 Sodium; AOBIOUS-AOB36447; stock solution prepared in 50% DMSO at a concentration of 10 mM) was added to the cells at different concentrations. After 24 h of incubation with the inhibitor, BRET measurements were performed by the addition of furimazine (Promega, Wisconsin, USA) at a dilution of 1:200. The percentage activity was calculated by normalizing the BRET ratio with the negative control (No M^{pro}). Two independent experiments were performed in triplicate for each sensor construct.

Western blot analysis. HEK 293T cells co-transfected with the M^{pro} sensor and the M^{pro} (wild-type or mutant) plasmids were lysed in 200 μl of 2× Laemmli sample buffer (50 mM Tris-Cl pH 6.8, 1.6% SDS, 8% glycerol, 4% $-mercaptoethanol and 0.04% bromophenol blue) (heated to 85° C. and sonicated prior to addition). Equal volumes of the cell lysates (30 μL) were separated by 10% SDS-PAGE using running buffer (25 mM Tris, 192 mM glycine, 0.1% SDS) at a constant voltage of 100 V for 1.5 h following which proteins were transferred onto PVDF (Polyvinylidene fluoride) membranes. Membranes were blocked in Tris-buffered saline containing 0.1% Tween-20 (TBS-T) with skimmed milk (5%) for 1 h at room temperature. Blots were incubated either with anti-His antibody (6×-His Tag Monoclonal Antibody (HIS.H8), Alexa Fluor 488; Thermofisher Scientific-MA1-21315-A488; 1:5000) or with anti-Strep-tag mouse monoclonal antibody (anti-Strep-tag mouse monoclonal, C23.21; PROGEN-910STR; 1:5000) overnight at 4° C. in dilution buffer (TBS-T containing 5% bovine serum albumin (BSA). Secondary anti-mouse IgG HRP (Anti-Mouse Ig:HRP Donkey pAb; ECM biosciences-MS3001; 1:10000 diluted in TBS-T) was used to detect M^{pro} and the cleaved M^{pro} sensor proteins.

Cloning, expression, and purification of M^{pro}-Nter-auto sensor in bacterial system. The mNG-M^{pro}-Nter-auto-NLuc was subcloned to pET-28b(+) plasmid using the restriction enzymes—HindIII and XhoI. The sensor was expressed in Escherichia coli (E. coli) BL21-CodonPlus cells (Agilent Technologies) in 100 mL of LB medium, as described previously. Protein expression was induced by the addition of 0.5 mM isopropyl-$-D-thiogalactopyranoside (IPTG), followed by overnight incubation at 20° C. After harvesting the cells by centrifugation (10000 g, 10 min, 4° C.), the pellet was resuspended in lysis buffer (10 mL per gram cell pellet; 10 mM phosphate buffer, 2.7 mM KCl, 507 mM NaCl, 10% glycerol, 20 mM imidazole and 0.1 mM DTT), followed by sonication. The supernatant was collected after centrifugation (18000 g, 90 min, 45° C.). The sensor construct was purified using Ni-NTA affinity chromatography. The concentration of the sensor was determined using Bradford assay.

Endpoint assay. Serial dilution of the purified sensor was prepared in buffer containing 50 mM HEPES, 50 mM NaCl, 0.1% Triton X-100, 1 mM Dithiothreitol (DTT) and 1 mM ethylenediamine tetraacetic acid (EDTA). Fifty (50) μL of sensor was incubated with 200 nM SARS-CoV-1 M^{pro} for 2:15 h at 37° C. The reaction was stopped by diluting each sample with TBS to a final concentration of 0.019 μM. BRET measurements were performed using a Tecan SPARK® multimode microplate reader.

Data Analysis and Figure Preparation. GraphPad Prism (version 9 for macOS, GraphPad Software, La Jolla California USA; www.graphpad.com), in combination with Microsoft Excel, was used for data analysis and graph preparation. Figures were assembled using Adobe Illustrator.

### Results and Discussion

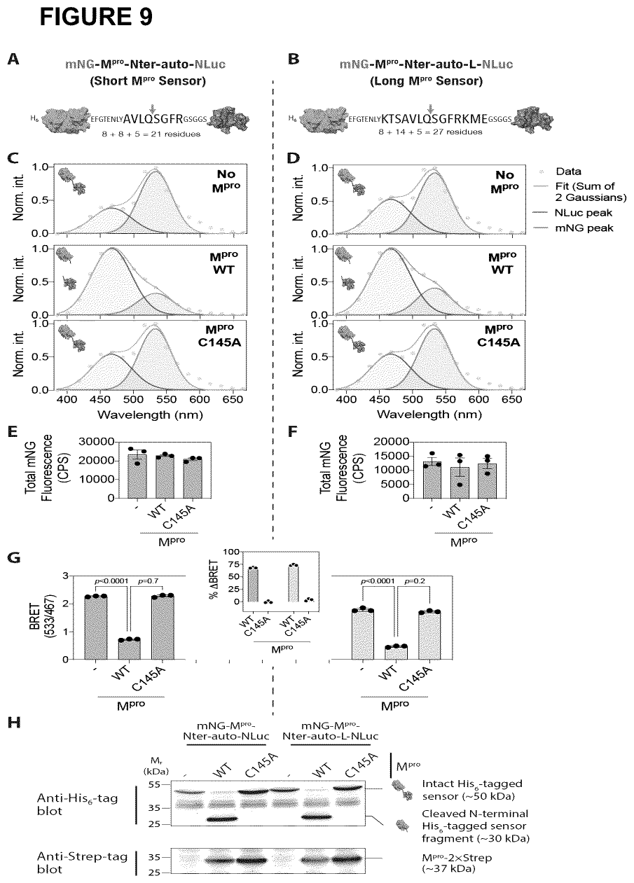

BRET-based M^{pro} proteolytic cleavage activity sensor design. In order to develop a live cell, BRET-based specific reporter to monitor M^{pro} proteolytic cleavage activity, fusion proteins were generated containing the M^{pro} N-terminal autocleavage sequence sandwiched between mNG (acceptor) and NLuc (donor) proteins (FIG. 6). The mNG and NLuc pair (acceptor and donor, respectively) has been used in a number of BRET-based sensors and show efficient energy transfer from NLuc to mNG. Thus, in the absence of any proteolytic cleavage, the sensor constructs are expected to display significant emission in the green channel. However, upon proteolytic cleavage of the sandwiched autocleavage peptide, the sensor constructs display reduced emission in the green channel with a concomitant increase in the emission in the blue channel (FIG. 6). Both SARS-CoV-210 and SARS-CoV-171 M^{pro} show a significant preference for the N-terminal autocleavage sequence (AVLQSGFR; short sensor [SEQ ID NO:1]; FIGS. 6, 9A) as a substrate compared to other cleavage sequences in the pp1a polyprotein in terms of catalytic efficiency, and has been widely utilized in FRET-based, in vitro assays as well as in a FlipGFP-based, live cell assay. Additionally, all available pp1a polyprotein sequences reported for SARS-CoV-2 isolates at the NCBI Virus database were analyzed for any variation in the cleavage sequence. This indicated that the N-terminal autocleavage sequence is invariable in all isolates reported and therefore, the sensor constructs described herein can serve as a general reporter for M^{pro} proteolytic cleavage activity. While BRET comes with several advantages including a higher signal-to-noise ratio and an extended dynamic range compared to some other methods, the presence of the acceptor and donor proteins i.e. mNG and NLuc at the N- and C-termini, respectively could potentially affect the interaction of the cleavage peptide with the M^{pro} dimer and thus, in turn, affect the cleavage efficiency of the peptide. This is especially relevant given that the binding of the peptide substrate has been reported to allosterically activate the SARS-CoV-1 M^{pro} dimer.

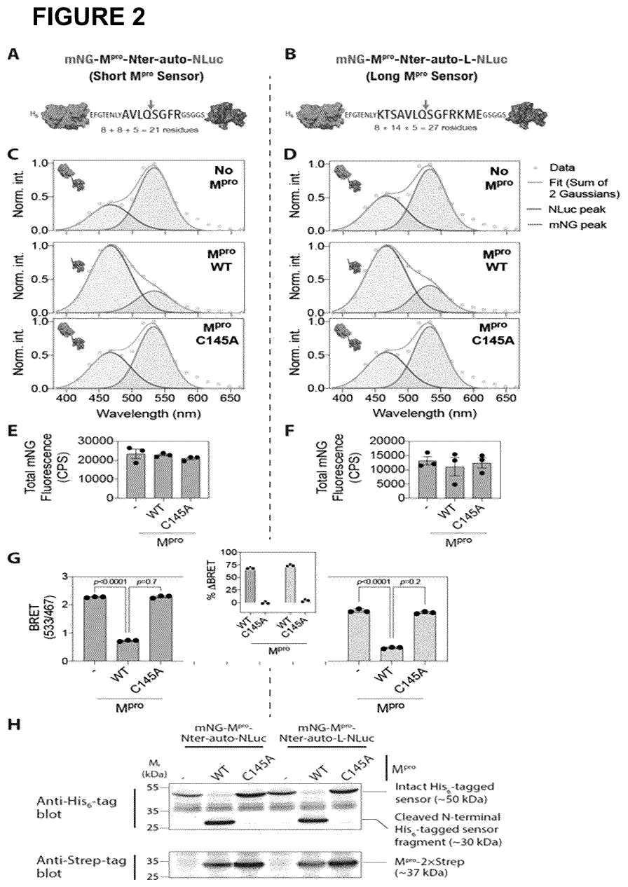

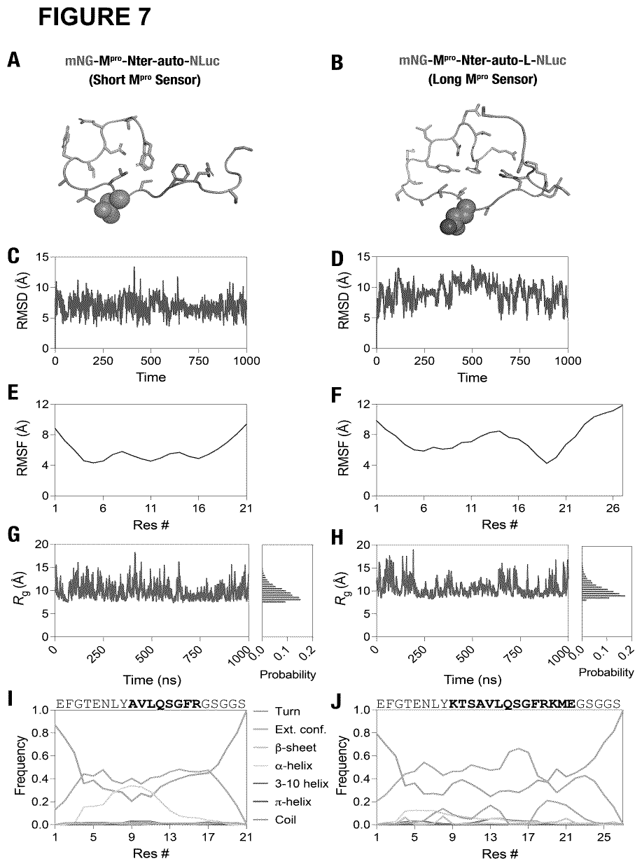

Therefore, a second, extended M^{pro} sensor construct was generated, the KTSAVLQSGFRKME [SEQ ID NO:2] peptide sequence (containing additional three residues on each side of the AVLQSGFR (SEQ ID NO: 1) core sequence; long sensor; SEQ ID NO:2; FIG. 2B). A key requirement for efficient cleavage of peptide substrates by M^{pro} is the structural flexibility of the peptide substrates. The formation of secondary structural element can alter cleavage activity, especially given that the secondary structure prediction indicated α-helical propensity by both the short as well as the long peptide (FIG. 8). In order to assess structural flexibility and secondary structure formation by the two peptides, structural models of the peptides were generated using the substrate peptide co-crystalized with the H41A mutant SARS-CoV-1 M^{pro} and performed all-atom, explicit solvent, Gaussian molecular dynamics (MD) simulation that allows enhanced sampling of protein conformational states. Structural models were generated using Modeler (FIGS. 7A, 7B) and MD simulations were performed using the NAMD software for a total duration of 1 μs for each peptide. These simulations indicated significant structural fluctuations in the two peptides as revealed by relatively large root-mean-squared-deviation (RMSD) and root-mean-squared-fluctuations (FIGS. 7C, 7D). Further, radius of gyration (R_{g}) measurements of the peptides over the course of simulation also revealed structural fluctuations of the peptides with an appreciably greater fluctuations observed for the short peptide compared to the long one (FIGS. 7G, 7H). The analysis further revealed a greater R_{g} for the longer peptide compared to the short peptide. Finally, a secondary structure analysis of the peptides over the course of the 1 μs long simulation trajectory revealed that the peptides largely show a propensity to form turns (FIGS. 7I, 7J). Notably, certain central residues in the shorter peptide can form α-helix that was not seen with the longer peptide leading to the possibility of a differential cleavage efficiency of the peptides by M^{pro}.

In the following, experimental results are reported with both the sensor constructs in order to provide a comparative analysis and determine the one that serves as a better substrate and thus, provide a superior evaluation of M^{pro} proteolytic cleavage activity in live cells.

FIGS. 7A-J show the M^{pro} N-terminal autocleavage peptide. M^{pro} N-terminal autocleavage peptide was found to be flexible. FIGS. 7A-B show the schematics of the M^{pro}-Nter-auto (short; FIG. 7A) and M^{pro}-Nter-auto-L (long; FIG. 7B) peptide structures modeled using the peptide substrate crystallized with H41A mutant SARS-CoV M^{pro} (PDB: 2Q6G). FIGS. 7C-D represent graphs showing backbone (Cα) root-mean-square deviation (RMSD) values of M^{pro}-Nter-auto (short; FIG. 7C) and M^{pro}-Nter-auto-L (long; FIG. 7D) peptide obtained from 1 μs of Gaussian MD simulations. FIGS. 7E-F represent graphs showing backbone (Cα) root-mean-square fluctuation (RMSF) values of M^{pro}-Nter-auto (short; FIG. 7E) and M^{pro}-Nter-auto-L (long; FIG. 7F) peptides. FIGS. 7G-H represent graphs showing radius of gyration (Rg) of the M^{pro}-Nter-auto (short; FIG. 7G) and M^{pro}-Nter-auto-L (long; FIG. 7H) peptides monitored over 1 μs of Gaussian MD simulations. FIGS. 7I-J represent graphs showing frequency of indicated secondary structures formed by the M^{pro}-Nter-auto (short; FIG. 7I) and M^{pro}-Nter-auto-L (long; FIG. 7J) peptides over 1 μs of Gaussian MD simulation.

BRET-based M^{pro} proteolytic cleavage activity sensor characterization in live cells. In order to test the functionality of the BRET-based M^{pro} proteolytic cleavage activity sensors, HEK 293T cells were transfected with the short and long sensors either alone or along with the M^{pro} expressing plasmid in a 1:5 sensor-to-protease plasmid ratio (FIG. 2). Additionally, the catalytic dead C145A mutant M^{pro} were utilized as a negative control in these experiments since Cys145 is essential for the proteolytic activity of M^{pro}. The transfection efficiency and expression of the sensor constructs was monitored by imaging lives cells for mNG fluorescence using an epifluorescence microscope, which showed an efficient transfection and expression of the sensor constructs after 24 h of transfection (FIG. 9). The spectral properties of the two sensors in live cells were then determined. For this, sensor construct transfected cells in adherent conditions were incubated with NLuc substrate and emission in the range of 380 nm and 664 nm wavelength were detected using a microplate reader. In the absence of co-expression of M^{pro}, both the short and the long sensors showed two peaks corresponding to NLuc (467 nm) and mNG (533 nm), respectively, as determined from two Gaussian fitting of the spectral data (FIGS. 2C, 2D; top panels). Co-expression of the wild type M^{pro} resulted in a decrease in the mNG emission peak in cells expressing either of the sensor constructs (FIGS. 2C, 2D; middle panels) while no such decrease was observed when the C145A mutant M^{pro} was co-expressed with the sensor constructs (FIGS. 2C, 2D; bottom panels). The co-expression of either the wild type or the C145A mutant M^{pro} did not result in any significant change in the intracellular levels of the sensor constructs as determined from mNG fluorescence at 530 nm under excitation with 480 nm light (FIGS. 2E, 2F). The BRET ratio of the sensor constructs in live cells under different M^{pro} protease co-expression conditions were then determined as a ratio of emission at 533 nm and 467 nm. Basal BRET ratio of the short and long sensors were found to be 2.37±0.17 vs 1.79±0.06 (mean±standard deviation; N=6 each; independent experiments performed in triplicates; p<0.0001), respectively, indicating that the additional 6 residues in the long sensor resulted in a 24 (±2) % decrease in the BRET ratio. Co-expression of the wild type M^{pro} resulted in a significant decrease in the BRET ratio while no significant decrease was observed in the presence of the C145A mutant M^{pro} (FIG. 2G). Importantly, both the short and the long sensor expressing cells showed ˜75% reduction in the BRET ratio in the presence of the wild type M^{pro} (FIG. 2G, inset graph), indicating that these sensors provide a wide dynamic range for monitoring M^{pro} proteolytic cleavage activity in live cells. Importantly, no change in the BRET ratio in cells expressing either of the sensors was observed in presence of the C145A mutant M^{pro} indicating high specificity of the BRET signals of these sensors (FIG. 2G, inset graph). In order to confirm that the reductions in the BRET observed upon co-expression with M^{pro}, western blot analysis were performed of cell lysates prepared from the M^{pro} sensor transfected cells. For this, the N-terminal His₆-tag in the M^{pro} sensor constructs were utilized, which were retained in the N-terminal, mNG protein containing fragment upon proteolytic cleavage, and C-terminal 2×Strep-tag in the M^{pro} protein for detecting cleavage of the M^{pro} sensor constructs and the expression of M^{pro}, respectively. Cells transfected with only the M^{pro} sensor constructs showed a band of the molecular weight of ˜50 kDa (as predicted from the amino acid sequence of the sensor constructs) (FIG. 2H). Indeed, cells co-expressing the wild type M^{pro}, as assessed from the anti-Strep-tag blot, showed a band of ˜30 kDa corresponding to the predicted molecular weight of the cleaved N-terminal fragment containing the His₆-tag and the mNG protein with a concomitant loss of the full-length sensor constructs (FIG. 2H). However, cells co-expressing the C145A mutant M^{pro}, as assessed from the anti-Strep-tag blot, did not show the cleaved sensor fragment (FIG. 2H). Agreement of these results with the BRET measurements shown above establishes that the reduction in the BRET ratio observed in the presence of the wild type M^{pro} is due to the proteolytic cleavage of the sensor constructs, and therefore, live cell BRET ratio measurements can be reliably used as a measure of M^{pro} proteolytic activity.

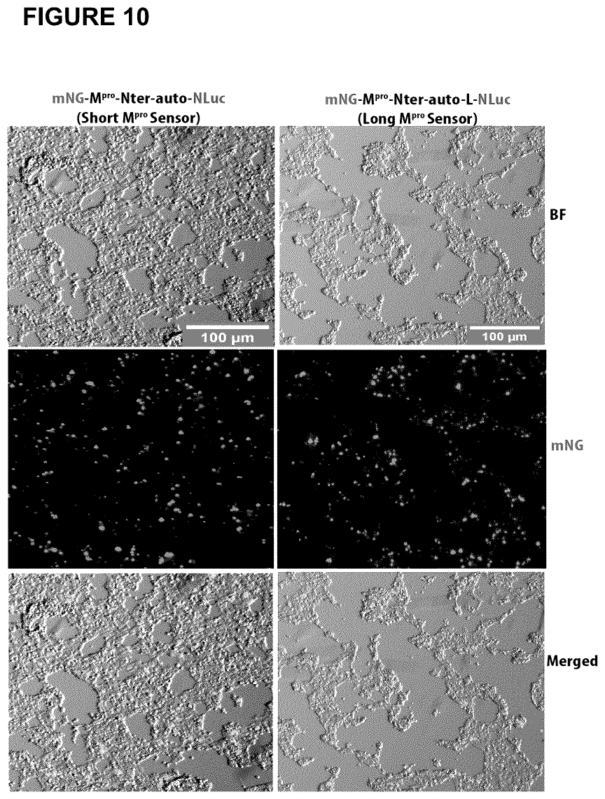

FIGS. 10A-B show M^{PRO} plasmid DNA dose-dependent cleavage of the M^{pro} sensors in live cells. FIGS. 10A-B depict graphs showing bioluminescence spectra of the short (FIG. 10A) and long (FIG. 10B) M^{pro} sensor constructs in cells expressing either the WT or C145A mutant M^{pro} protease. Data were fit to a two Gaussian model reflecting mNG fluorescence and NLuc bioluminescence peaks. Note the dose-dependent cleavage of both the short (FIG. 10A) as well as the long (FIG. 10B) sensors specifically in the presence of the WT M^{pro} as reflected by the reduction in the mNG peak (533 nm) of the sensor constructs.

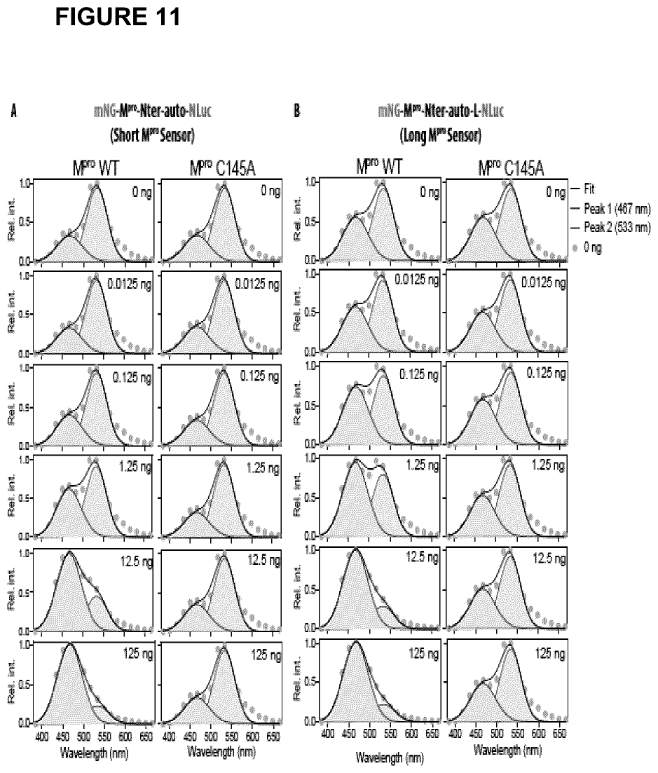

M^{pro} dose-dependent cleavage of the sensor in live cells. Having established that the BRET ratio could be used to detect M^{pro} proteolytic activity of the sensor constructs, the M^{pro} dose-dependent cleavage of the sensor constructs in live cells was determined. For this, the cells with the 25 ng/well sensor constructs were co-transfected and a range of M^{pro} plasmid concentrations (0, 0.0125, 0.125, 1.25, 12.5 and 125 ng/well) and monitored bioluminescence spectra in adherent cells after 48 h. This revealed a M^{pro} plasmid dose-dependent shift in the bioluminescence spectra (FIGS. 11A-B) and the BRET ratio (FIGS. 11C-D) of both the short and the long sensor in the presence of the wild type M^{pro} but not in the presence of the C145A mutant M^{pro}. Discernable decreases in the BRET ratio could be observed at a minimum amount of 1.25 ng of M^{pro} plasmid DNA and a maximum decrease in the BRET ratio of ˜80% at the highest concentration of 125 ng for both sensor constructs (FIGS. 11C-D; inset graphs). The analysis has also showed that the EC₅₀ values are 1.09±0.09 ng/well and 0.91±0.89 ng/well for the short and the long sensors, respectively. These data demonstrate the functional potency of M^{pro} expressed in these cells.

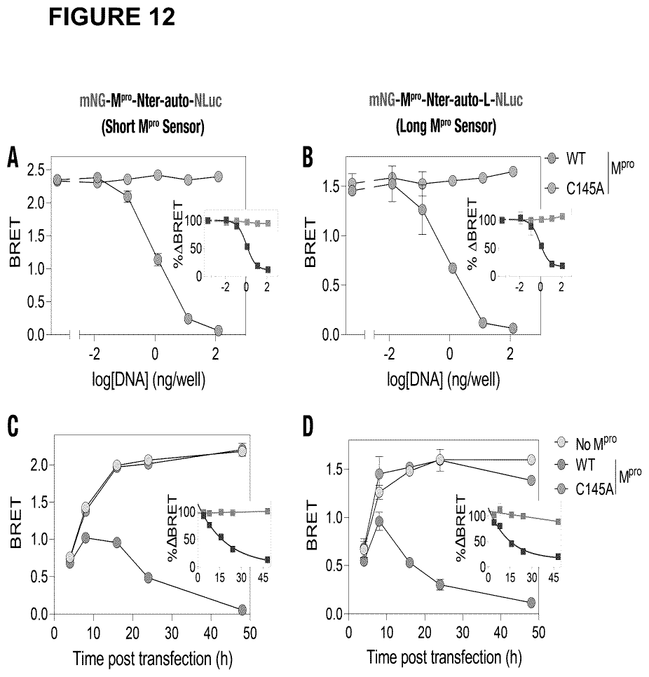

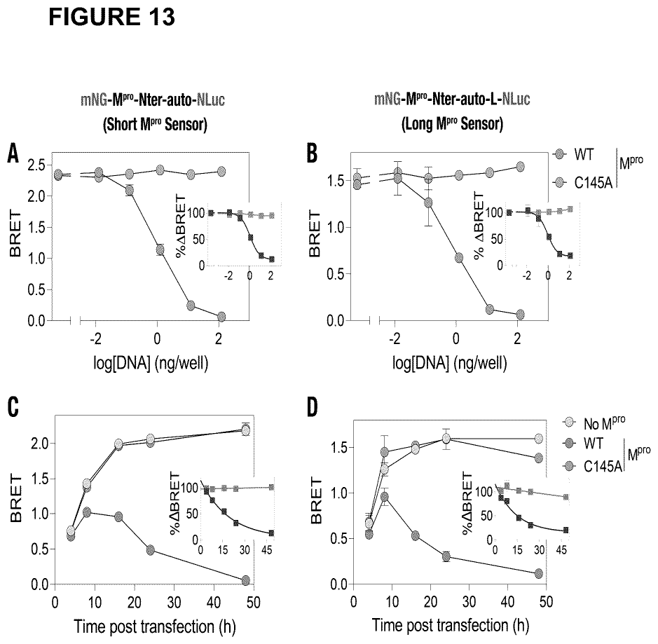

Monitoring the temporal dynamics of M^{pro} protease activity in live cells. The temporal dynamics of M^{pro} proteolytic activity in live cells were monitored. Towards this, the cells were transfected with the M^{pro} sensor constructs either in the absence or in the presence of the wild type or the C145A mutant M^{pro} plasmid and monitored the bioluminescence spectra from 4 h post transfection (FIGS. 13A, 12B). Analysis of the bioluminescence spectra obtained from cells expressing either of the M^{pro} sensors indicated a lower BRET ratio after 4 h of transfection, which increased with time and plateaued after 16 h of transfection in the absence of M^{pro} (FIGS. 13C-D). Although mNG shows a relatively fast maturation time compared to several other fluorescent proteins, these data likely indicate a relatively slower intracellular maturation of mNG compared to NLuc. Importantly, a significant decrease in the BRET ratio of cells expressing either of the M^{pro} sensors could be observed in the presence of the wild type M^{pro} after 8 h of transfection (FIGS. 13C-D). BRET ratio of the cells continued to decrease in the presence of the wild type M^{pro} until 48 h of transfection while no such decrease was observed in the presence of the C145A mutant M^{pro} (FIGS. 8C-D; inset graphs). The half-life of the protease was found to be 13.22±3.22 h and 9.81±2.54 h for the short and the long sensors, respectively. These data suggests that the BRET-based M^{pro} sensors described herein can report M^{pro} proteolytic activity as early as 8 h of infection. This can vary depending on the actual expression of the protease in host cells.

FIGS. 12A-B show temporal dynamics of M^{pro} protease activity in live cells. FIGS. 12A-B represent graphs showing bioluminescence spectra of the short (FIG. 12A) and the long (FIG. 12B) M^{pro} sensor constructs either in control cells or in cells expressing the WT or C145A mutant M^{pro} protease. Data were fit to a two Gaussian model reflecting mNG fluorescence and NLuc bioluminescence peaks. Note the time-dependent cleavage of short (FIG. 12A) and long (FIG. 12B) sensors specifically in the presence of the WT M^{pro} as reflected by the reduction in the mNG peak (533 nm) of the sensor constructs.

FIGS. 13A-D show a time-dependent expression of the BRET-based M^{pro} sensors. FIGS. 13A and 13C represent epifluorescence images acquired using a 4× objective of HEK 293T cells transfected with either pmNG-M^{pro}-Nter-auto-NLuc (short; FIG. 13A) or pmNG-M^{pro}-Nter-auto-L-NLuc (long; FIG. 13C) plasmids showing a time-dependent increase in the number of cells expressing the sensors. FIGS. 13B and 13D represent graphs showing time-dependent increase in GFP⁺ cells after transfection with either pmNG-M^{pro}-Nter-auto-NLuc (short; FIG. 13B) or pmNG-M^{pro}-Nter-auto-L-NLuc (long; FIG. 13D) plasmids.

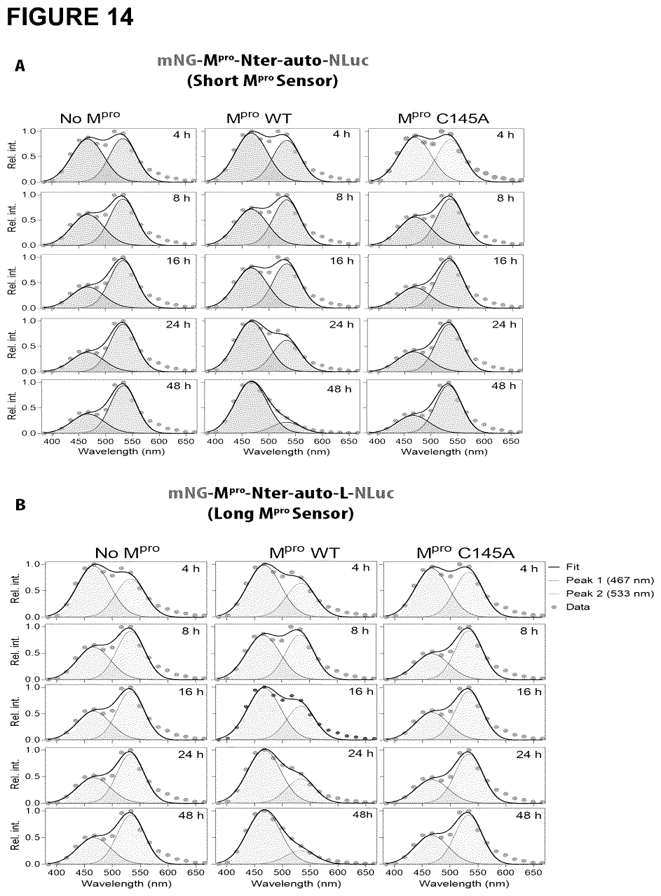

Comparison with the FlipGFP-based M^{pro} proteolytic sensor in live cells. Having established the monitoring of expression-dependent proteolytic activity of M^{pro} in live cells, similar experiments were performed with FlipGFP-based M^{pro} proteolytic activity reporter in order to compare the performance of the biosensors in reporting M^{pro} proteolytic activity in live cells. Accordingly, HEK 293T cells were transfected with the FlipGFP M^{pro} sensor expression plasmid along with either the WT or C145A M^{pro} expression plasmid and monitored GFP expression in the cells to ascertain conversion of the non-fluorescent protein to a fluorescent one while mCherry expression in the cells was used for detecting transfected cells. Epifluorescence imaging of the cells post 4 h of transfection revealed the appearance of GFP fluorescence in the transfected cells, as ascertained from mCherry fluorescence, in the presence of WT M^{pro} after 24 h of transfection (48±2%) while more cells showed GFP⁺ fluorescence after 48 h of transfection (70±4%) (FIGS. 14A-B). The resulting data indicated a delayed response of the FlipGFP sensor to M^{pro} proteolytic activity in comparison to the BRET-based sensor. Additionally, a significant number of cells were found to be GFP positive after 24 h (9±1%) and 48 h (20±1%) of FlipGFP transfection in the presence of the C145A mutant M^{pro} (FIGS. 14C-D). This is contrast to the observations made with the BRET-based sensor in the presence of the mutant M^{pro} (FIGS. 13C-D).

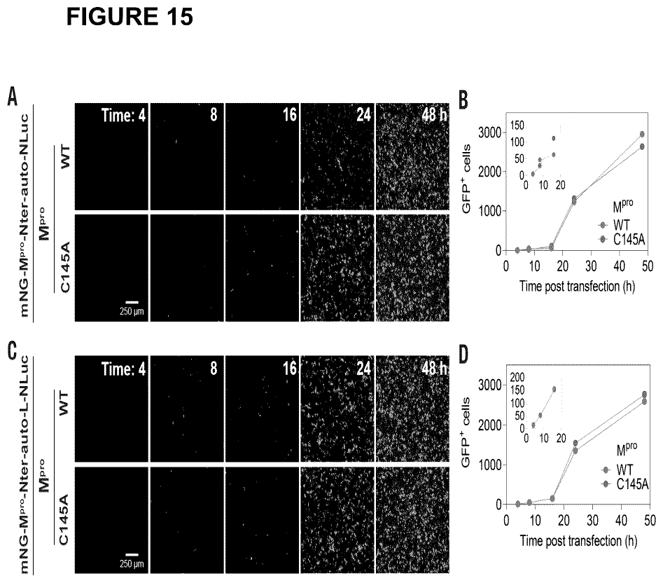

FIGS. 15A-B show the GC376-mediated M^{pro} inhibition monitored in live cells and the bioluminescence spectra of the ne-No M^{pro} control. FIGS. 15A-B depict graphs showing bioluminescence spectra of the short (FIG. 15A) and long (FIG. 15B) M^{pro} sensor constructs in cells treated with the indicated concentrations of GC376 inhibitor in cells co-expressing either the WT or the C145A mutant M^{pro} protease. The bioluminescence spectra of the No M^{pro} control is also shown. Data were fit to a two Gaussian model reflecting mNG fluorescence and NLuc bioluminescence peaks. Note the dose-dependent inhibition of protease activity of WT M^{pro} in cleaving both the short (FIG. 15A) as well as the long (FIG. 15B) sensors which is evident from the increased intensity of mNG peak (533 nm) of the sensor constructs.

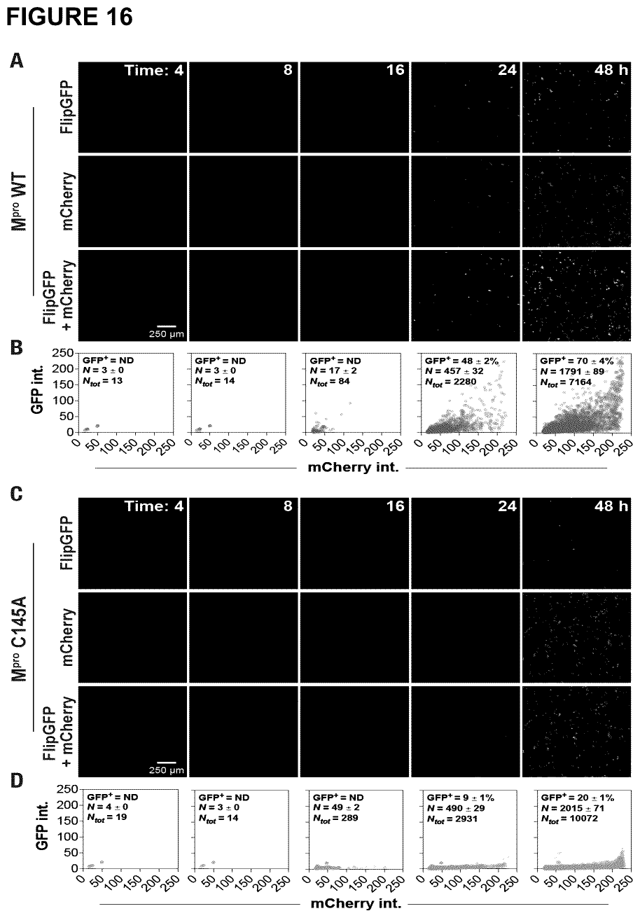

Monitoring pharmacological inhibition of M^{pro} proteolytic activity in live cells. Finally, the utility of the BRET-based M^{pro} sensors in pharmacological inhibition of M^{pro} proteolytic activity in live cells was determined. Towards this, cells co-expressing the M^{pro} sensors and M^{pro} were treated with various concentrations of GC376, which has been shown to inhibit M^{pro} in live cells after 8 h of transfection based on the results reported above, and determined bioluminescence spectra of the cells after an additional 24 h (FIGS. 16A-B). A GC376 dose-dependent increase in the BRET ratio of cells co-expressing either the short or the long sensor and the wild type M^{pro} was observed, while no sensor cleavage was observed in the presence of the C145A mutant M^{pro} (FIGS. 16C-D). Percentage proteolytic cleavage activity determined from the BRET ratio indicate that GC376 starts to inhibit M^{pro} at 33.3 μM concentration and continued to do so until a concentration of 333 μM (FIGS. 16C and 16D; inset graphs). The data demonstrates that the IC₅₀ values for the short sensor is 127.4±23.33 μM and that for long sensor is 194.7±7.49 μM. The lower efficacy of GC376 observed here compared to previous reports can indicate a cell type- or M^{pro} expression-dependent effect. Taken together, these data indicate that the BRET-based M^{pro} proteolytic activity sensors described herein can be utilized for screening antivirals targeted against M^{pro}.

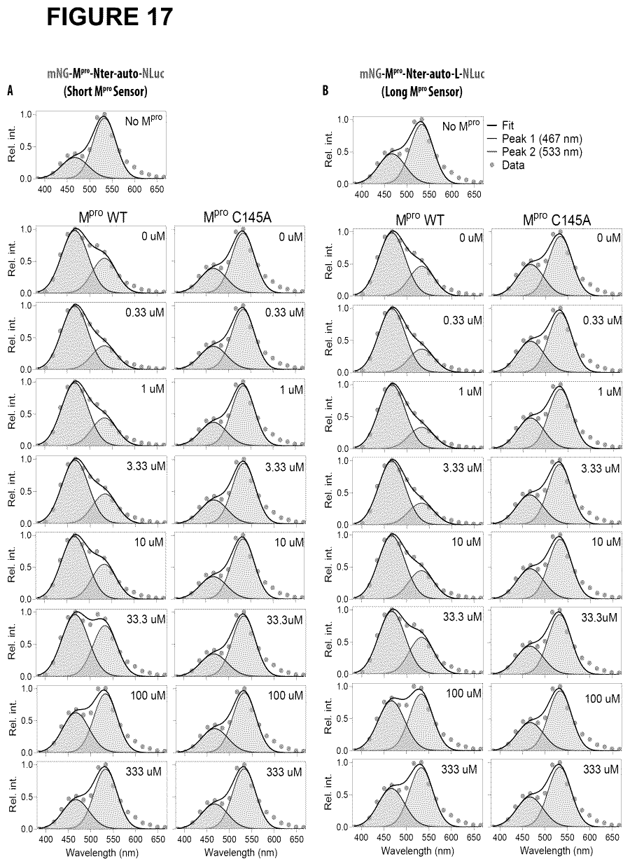

Monitoring M^{pro} proteolytic cleavage activity in vitro. Having established the utility of the BRET-based M^{pro} sensor in live cell studies, determination of the BRET-based M^{pro} sensor utility in vitro was carried out using a recombinantly purified SARS-CoV-1 M^{pro}. Lysates were prepared from HEK 293T expressing either the short or the long M^{pro} sensor construct, incubated equivalent amounts of the lysates with three different concentrations (5 μM, 500 nM and 50 nM) of the recombinantly purified M^{pro} and monitored BRET following addition of the NLuc substrate (FIGS. 17A-B). The assay revealed an M^{pro} concentration-dependent proteolytic processing of the M^{pro} sensors as ascertained from the decreasing BRET ratios. Importantly, the assay also indicated that a minimum of 500 nM of the recombinantly purified M^{pro} is required for a discernable proteolytic cleavage of the sensors as the BRET ratio of the sensors decreased to a lesser extent in the presence of 50 nM M^{pro} protein while a substantially higher rate of cleavage was observed under 500 nM M^{pro}. The assays were then performed in the presence of GC376 to determine the pharmacological inhibition of M^{pro} activity in vitro. Accordingly, 500 nM M^{pro} was preincubated with a range of concentrations of GC376 (10-4 to 10-9 M) for 30 minutes at 37° C. and cleavage activity was monitored after addition of lysates prepared from cells expressing either the short or the long M^{pro} sensor. Incubation with GC376 resulted in a decrease in the rate of proteolytic cleavage of both the short and the long M^{pro} sensor (FIGS. 17C-D) with IC₅₀ values of 73.1±7.4 and 86.9±11.0 nM for the short and the long sensor, respectively (FIGS. 17G-H).

In vitro assays reveal molecular crowding-mediated increase in M^{pro} proteolytic activity and a decrease in inhibitor efficacy. The slow rate of the M^{pro} sensor cleavage under 500 nM M^{pro} was used to determine the effect of molecular crowding on the proteolytic activity of the protein. Molecular crowding in the intracellular environment caused by the presence of soluble and insoluble macromolecules such as proteins, nucleic acids, ribosomes and carbohydrates has been shown to impact both structure and stability of proteins in cells as well as enzyme kinetics including a decrease in the activity of hepatitis C virus (HCV) NS3/4A protease and an increase in the proteolytic activity of SARS-CoV M^{pro}. Twenty-five (25)% (weight/volume; w/v) of 20000 Da (20K) polyethylene glycol (PEG) was included, which is a non-toxic, hydrophilic polyether that serves as a crowding agent and has been extensively utilized to simulate the molecular crowding in vitro, in the assays and monitored cleavage of the M^{pro} sensors under 500 nM M^{pro} under varying concentrations of GC376. Inclusion of 25% PEG 20K resulted in a substantial increase in the rate of proteolytic cleavage of the M^{pro} sensors in the absence of GC376 (FIGS. 17E-F). The resulting data suggests that molecular crowding caused by PEG 20K is likely effective in causing increased dimerization of M^{pro}, a feature critical for its catalytic activity, through an increase in the effective concentration of the protein due to excluded volume effects, and thus increases the rate of proteolytic cleavage of the M^{pro} sensor. Importantly, while GC376 could inhibit M^{pro} activity, the IC₅₀ values as obtained from BRET ratios after 2 h of incubation with both the short as well as the long sensor indicated a large shift (IC₅₀ values of 2623±760 and 10260±3280 nM, respectively) (FIGS. 17G-H). Suitably, the data indicate that M^{pro} can be more active in the crowded environment of an infected host cell compared to in vitro conditions; and can require higher concentrations of pharmacological inhibitors for effective inhibitions of it catalytic activity than those determined from in vitro assays.

Dynamic Light Scattering (DLS) measurements. DLS measurements of recombinantly purified proteins were performed using the Zetasizer Nano ZS (Malvern Panalytical, Malvern, United Kingdom). Proteins, either bovine serum albumin (BSA; Tocris Bioscience, Cat. No. 5217) or the short M^{pro} sensor, were prepared in 1×TBS at a final concentration of 1 μM and 400 nM, respectively and light scattering measurements were performed. The purified short M^{pro} biosensor protein was centrifuged prior to measurement at 14000 rpm for 1 hour at 4° C. and supernatant taken to remove any aggregates. Multiple size spectra (N=40 for BSA and 61 for the short M^{pro} biosensor) obtained from triplicate measurements for 5 s were used for the determination of average molecular size of the proteins.

In vitro enzyme kinetics measurements. To determine the initial reaction velocity, a range of the short M^{pro} sensor concentrations were incubated with 200 and 500 nM of the recombinantly purified M^{pro} protein in a buffer containing 50 mM HEPES, 50 mM NaCl, 0.1% Triton X-100, 1 mM 1 mM Dithiothreitol (DTT) & 1 mM ethylenediamine tetraacetic acid (EDTA) in a total volume of 50 μL for 2.25 h at 37° C. Following incubation, each reaction was diluted to a final concentration of 20 nM of the M^{pro} sensor and BRET measured using a Tecan SPARK® multimode microplate reader after addition of NLuc substrate. For initial reaction velocity calculation at each sensor (substrate) concentration, a background BRET value of 0.25 (obtained using NLuc alone) was subtracted from the initial as well as final BRET values. Reaction rates were then calculated as:

$$Rate = [ ( {BRET}^{Initial} - {BRET}^{Final} ) \times [ {M}^{pro} sensor ] / time$$

$$where Rate = reaction velocity at a {M}^{pro} sensor concentration$$

$${BRET}^{Initial} and {BRET}^{Final} = initial and final BRET ratio , respectively ,$$

$$[ {M}^{pro} se nsor ] = concentration of {M}^{pro} sensor$$

$$time = incubation time$$

To conclude, genetically encoded, BRET-based M^{pro} protease activity sensors have been developed for use in live cells and their utility for antiviral drug discovery has been validated using GC376 as a proof of principle. The use of BRET, with NLuc as the bioluminescence donor and mNG as the resonance energy acceptor, enabled highly sensitive detection of M^{pro} protease activity in live cells. Additionally, the sensors developed here did not show any cleavage, either in the absence of M^{pro} or in the presence of the catalytically dead, C145A mutant M^{pro}, thus, displaying relatively high specificity. In some embodiments, these sensors have utility in both detecting active SARS-CoV-2 infection as well as in screening antivirals developed for targeting M^{pro} proteolytic cleavage activity in live cells. Additionally, in some embodiments, they can be utilized for determining effects of genetic variation in the M^{pro} amino acid sequence that can arise during the evolution of the virus.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A sensor, comprising J¹ connected by a linker to J², wherein:
the linker comprises an M^{pro} peptide sequence;
J¹ comprises a NanoLuc peptide sequence; and
J² comprises an mNeonGreen peptide sequence.

2. The sensor of claim 1, wherein the M^{pro} peptide sequence comprises an M^{pro} cleavage peptide sequence.

3. The sensor of claim 1, wherein the M^{pro} peptide sequence is AVLQSGFR (SEQ ID NO:1).

4. The sensor of claim 1, wherein the M^{pro} peptide sequence is KTSAVLQSGFRKME (SEQ ID NO:2).

5. The sensor of claim 1, wherein the sensor comprises the peptide sequence EFGTENLYAVLQSGFRGSGGS (SEQ ID NO:3) or EFGTENLYKTSAVLQSGFRKMEGSGGS (SEQ ID NO:4).

6. The sensor of claim 1, wherein the linker further comprises an M^{pro} protease peptide sequence.

7. A composition, comprising the sensor of claim 1 and an excipient.

8. A method of determining M^{pro} proteolytic inhibition of a compound, comprising contacting the compound with an M^{pro} peptide sequence having protease activity in the presence of the sensor of claim 1.

9. The method of claim 8, further comprising measuring a fluorescence emission of the sensor and comparing the fluorescence emission with an initial fluorescence emission of the sensor prior to contact with the compound.

10. A method of determining protease activity of an M^{pro} peptide sequence, comprising contacting the sensor of claim 1 with the M^{pro} peptide sequence.

11. The method of claim 10, further comprising measuring a fluorescence emission of the sensor and comparing the fluorescence emission with an initial fluorescence emission of the sensor prior to contact with the M^{pro} peptide sequence.

12. An oligonucleotide sequence, coding for the sensor of any of claims 1-6.

13. A vector, comprising the DNA sequence of claim 12.
